(19) United States
(12) Patent Application Publication
(10) Pub. No.: US 2007/0239094 A1
(43) Pub. Date: Oct. 11, 2007

(54) Ingrown toenail treatment method

(76) Inventor: Byung Cha, New Brighton, MN (US)

Correspondence Address: Byung Kwon Cha, Suite 4, 1401 Silver Lake Road, New Brighton, MN, 55112, US

(21) Appl. No.: 11/398,011

(22) Filed: Apr. 5, 2006

(51) Int. Cl. A61F 5/00

(52) U.S. Cl. 602/31

(57) ABSTRACT

A method for treating an ingrown toenail by providing a smooth surface on the lateral edge of the damaged/sharpened nail and isolating the lateral nail bed, i.e., the cuticle, from the sharpened nail, thereby aborting the cycle of mutual damage that occurs when in ingrown toenail causes inflammation under the toenail which in turn then further dissolves the nail edge, which in turn then causes further inflammation. By ceasing this cycle, the sufferer of the ingrown toenail feels immediate pain relief while the toenail heals itself by means of isolation of the cuticle from the sharpened portion of the nail.

### PRIOR APPLICATION

This application emanates from a prior Provisional Patent Application filed May 26, 2005.

### FIELD OF THE INVENTION

This invention relates to the field of treatment for the condition of ingrown toenails. In particular, this invention involves a method that a person can use without the assistance of medical personnel.

### BACKGROUND OF THE INVENTION

An ingrown toenail occurs when part of the toenail penetrates the fleshy part of the skin. This often results in an infection. Usually one side of the nail penetrates deep into the toe, making it difficult to see the edge of the nail. The corner or a small spike of nail penetrates the skin, just like a knife. This can result in an infection and the development of proud flesh, i.e., granulation tissue. The toe will then be red, inflamed and painful.

Pain is the main symptom of an ingrown toenail, usually starting out as just minor discomfort. This may be just the pressure from the side of the nail or it may be because the nail has actually penetrated the skin down the side of the nail. The toe is not necessarily infected, but an infection can develop after the nail penetrates the skin and becomes ingrown. The infection can then spread, making the toe red and inflamed. A collection of pus may also develop.

Poor cutting of the nail is the most commonly held belief as to the cause of ingrown toenails, but other factors such as poorly fitted shoes, infections and other traumas are also believed to cause ingrown toenails. The cornerstone of self-treatment and prevention of ingrown toenails involves the cutting of the nail straight across the width of the nail, allowing the corners to protrude, ostensibly so that they do not penetrate the skin. The toenails are typically cut straight across without tapering the corners. However, this is not always easy, especially if the nail is very curved down the side.

It is a myth that a “V” should be cut in the end of the nail to treat an ingrown toenail. The apparent reasoning behind this is that if you cut a “V” in the nail, the edge of the nail will grow together as the nail grows out. This does not actually occur, as the growing area at the base of the toe rather than at the end determines the shape of the nail.

Existing efforts to treat ingrown toenails involve the restoration of the shape of the toenail. However, those efforts have not been successful in all cases because the misshaped toenail is not the real cause of the pain. The infection and cycle of pain and damage that occurs once the condition has started has little to do with the shape of the nail. The ingrown toenail starts with damage to the lateral nail bed, i.e., the cuticle that often develops into a sharpened edge. The damaged cuticle then produces an inflammatory reaction in the toe. In turn, the matter produced by the inflammation further dissolves the lateral side of the nail, making the edge even sharper, thereby producing further cutting into the skin, further damaging the cuticle and causing further infection and inflammation. Unless there is an interruption in this cycle of mutual damage, the problem will steadily worsen.

Prior art in the field addresses the theory that reshaping the nail will solve the problem. Vironda in U.S. Pat. No. 3,981,298 describes a system wherein a liquid material is applied to a nail in a limited area, which hardens and then serves as a form-retaining support. This invention involves the application of a strip that spreads along the entire width of the nail. However, this invention only restores the shape of the nail and does not address the cycle of inflammation and nail damage that is occurring at the site of the inflammation. Steele in U.S. Pat. No. 5,862,811 defines a system that involves an adhesive and solvent that fills cracks in nails with polystyrene pellets. Like the Vironda invention, the Steele invention does not seek to isolate the problem at the damaged point of the nail, but merely seeks to reshape the nail in hopes that it will grow out of the embedded fleshy portion of the toe. Similarly, Machida in U.S. Pat. No. 6,095,995 teaches of a plate-like member that is made from a shape-memorizing alloy or resin material that is designed to match the curvature of the nail at room temperature which then deforms into a memorized flat shape when heated.

As with the previous inventions, Machida seeks only to reshape the nail. Accordingly, there exists a need for a system that isolates the sharpened edge of the nail and provides a smooth surface on the lateral edge of the nail, thereby reducing the cycle of inflammation, infection, damage and pain.

### SUMMARY OF THE INVENTION

In the preferred embodiment of the invention, a method is described in the following steps. First the toe with the ingrown toenail is soaked in warm water to soften it, for approximately ten minutes. The toe is then cleaned with anti-bacterial soap and then completely rinsed with clean water. The toe is then dried with a towel. The toe is then disinfected on the side of the damaged portion as well as underneath it. This is accomplished through the application of an anti-microbial substance that is absorbed into a cotton ball. The cotton ball is guided with a toothpick to the affected area.

Using a finger, the damaged side of the nail bed is pushed outward and a non-toxic liquid bonding agent is then applied to cover the entire affected area, both underneath the toenail and on the side of the toenail. The bonding agent then must be dried. This can be accomplished through ambient temperature or can be accelerated through the use of a hair dryer, or other mechanical heating means. Once the bonding agent has dried, the pain should immediately cease. The toenail should then be checked daily and washed with soap. The anti-microbial agent should also be re-applied daily. If the bond between the nail and the side of the nail bed becomes separated, the bonding agent should be re-applied. Once the portion of the nail containing the bonding agent grows to a sufficient length, the nail can then be clipped and discarded.

### OBJECT OF THE INVENTION

The principal object of the invention is to provide a smooth surface on the lateral edge of the damaged/sharpened nail and isolating the lateral nail bed, i.e., the cuticle, from the sharpened nail, effectively stopping the cycle of mutual damage occurring between the sharpened edge of the nail and infected fleshy portion of the toe.

### BRIEF DESCRIPTION OF THE DRAWINGS

FIG. 1 illustrates a top view of a toe affected with an ingrown toenail.

FIG. 2 illustrates a top view of an affected toe being treated with an anti-microbial substance using a cotton ball and a toothpick.

FIG. 3 illustrates a top view of an affected toe with a finger pushing the affected portion of the toenail outward.

FIG. 4 illustrates a top view of an affected toe with liquid bonding agent applied to the affected area.

### DETAILED DESCRIPTION OF AN ENABLING AND PREFERRED EMBODIMENT

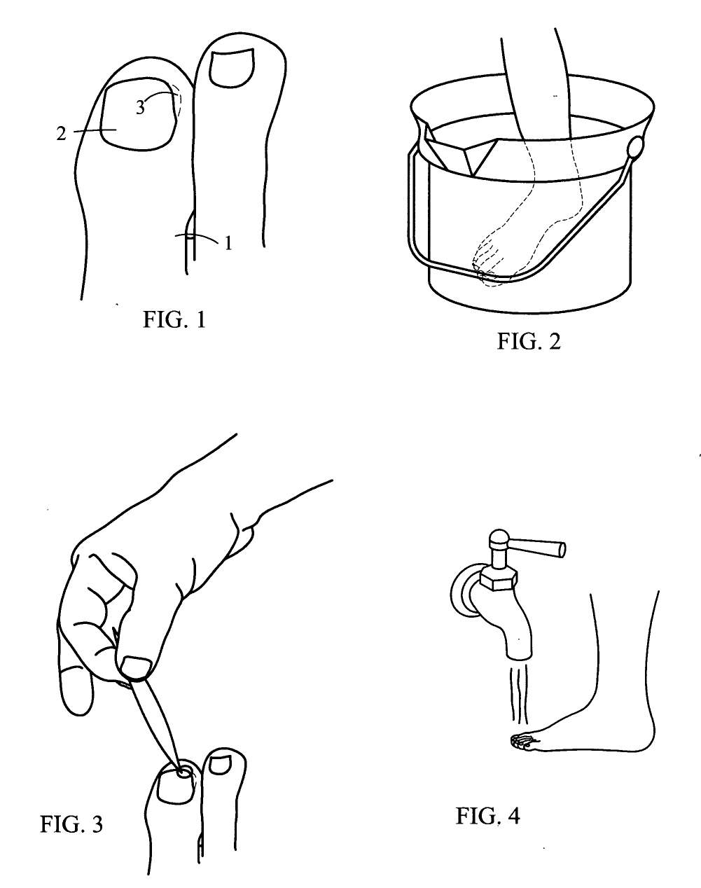

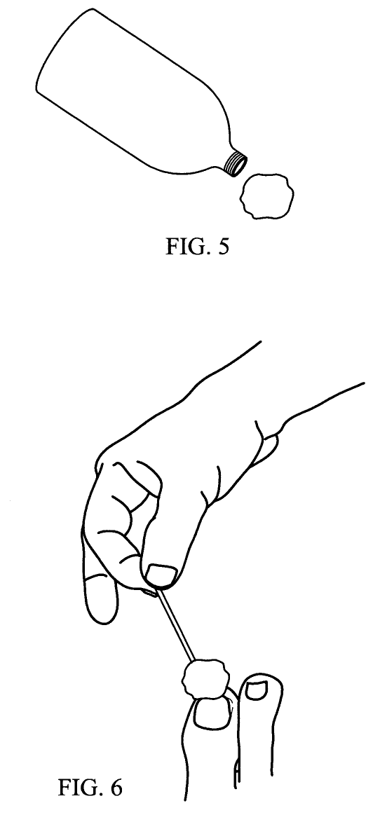

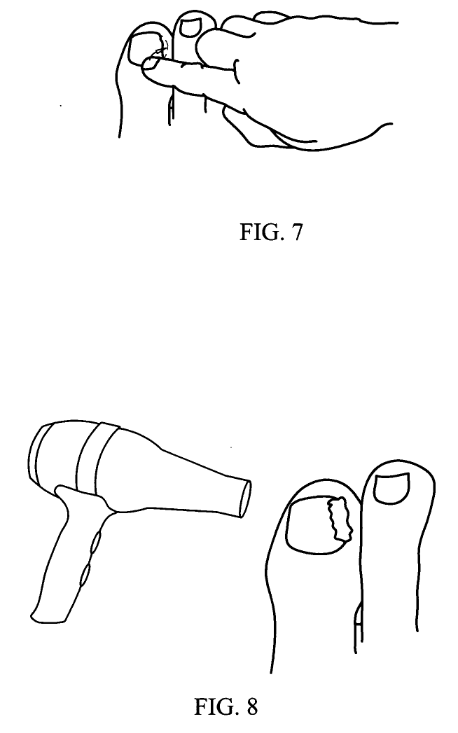

For a better understanding of the invention, we turn now to the drawings. FIG. 1 offers a visual understanding of how an ingrown toenail appears to the eye. The toe 1 has a nail 2 that has a portion 3 that is cutting into the flesh of the skin and creating an infection. To treat this condition, first the toe 1 is soaked in warm water to soften it, cleaned with water, rinsed and then dried (not shown). FIG. 2 shows the toe 1 then being disinfected on the side and the underneath of the ingrown toenail 3 through the application of an anti-microbial substance that is absorbed into a cotton ball 5. The cotton ball 5 is then guided with a toothpick 4 to the affected area 3.

FIG. 4 shows the use of a finger 6 to push the damaged side of the nail 3 outward. FIG. 4 shows where the liquid bonding agent 7 has been applied to the damaged area.

The illustrations and examples provided herein are for explanatory purposes and are not intended to limit the scope of the appended claims, as those skilled in the art will make modifications to the invention for particular uses.

I claim:

1. A method for treating an ingrown toenail comprising
soaking a toe suffering from an ingrown toenail in water for a sufficient amount of time to soften said toe;
cleaning said toe with a cleaning agent;
thoroughly rinsing said toe with clean water after said cleaning;
drying said toe after said rinsing;
soaking an anti-microbial substance onto an absorbent means;
applying said anti-microbial substance-soaked absorbent means through the use of a guiding means to said toe, applying said anti-microbial substance over the entirety of said toe;
gently pushing the damaged portion of said ingrown toenail outward with a pushing means;
applying a bonding means to cover the affected area of said toe, coating the underneath and side of said ingrown toenail;
allowing said bonding means to dry;
continuing to wash said toenail daily and applying said anti-microbial substance until said toenail has grown to a sufficient length to be clipped off and discarded;
re-applying said bonding agent as needed until said ingrown toenail has grown, been clipped and discarded.

2. A method according to claim 1 wherein said toe is soaked in water for 10 ten minutes at the first step.

3. A method according to claim 1 wherein said cleaning agent is an anti-bacterial soap.

4. A method according to claim 1 wherein said toe is dried using a towel.

5. A method according to claim 1 wherein said absorbent means is a cotton ball.

6. A method according to claim 1 wherein said guiding means is a toothpick.

7. A method according to claim 1 wherein said pushing means is a finger.

8. A method according to claim 1 wherein said bonding means is a non-toxic liquid bonding agent.

9. A method according to claim 1 wherein said bonding agent is dried at room temperature.

10. A method according to claim 1 wherein said bonding agent is dried through a mechanical means, such as a hairdryer.
